(19) United States
(12) Patent Application Publication
(10) Pub. No.: US 2006/0264469 A1
(43) Pub. Date: Nov. 23, 2006

(54) PHARMACEUTICAL COMPOSITION FOR ORAL ADMINISTRATION OF A PYRAZOLE-3-CARBOXAMIDE DERIVATIVE

(75) Inventors: Thierry Breul, Frontignan (FR); Jean-Claude Gautier, Clapiers (FR); Olivier Saslawski, Montpellier (FR)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: Sanofi-aventis, Paris (FR)

(21) Appl. No.: 11/382,111

(22) Filed: May 8, 2006

Related U.S. Application Data
(63) Continuation of application No. PCT/FR04/02875, filed on Nov. 9, 2004.

(30) Foreign Application Priority Data
Nov. 10, 2003 (FR) 0313259

(51) Int. Cl. A61K 31/454

(52) U.S. Cl. 514/326

(57) ABSTRACT

This invention discloses and claims a pharmaceutical composition in liquid or semi-liquid form, which is self-emulsifying or self-microemulsifying in aqueous medium, for the oral administration of a pyrazole-3-carboxamide derivative, in which said derivative is dissolved in an amphiphilic mixture containing one or more lipid solvents and a nonionic hydrophilic surfactant.

### CROSS REFERENCE TO RELATED APPLICATIONS

This application is a continuation of International application No. PCT/FR2004/002,875, filed Nov. 9, 2004, which is incorporated herein by reference in its entirety; which claims the benefit of priority of French Patent Application No. 03/13,259, filed Nov. 10, 2003.

### BACKGROUND OF THE INVENTION

1. Field of the Invention

The present invention relates to a pharmaceutical composition for the oral administration of a pyrazole-3-carboxamide derivative, and of its pharmaceutically acceptable salts and the solvates thereof.

2. Description of the Art

The term “pyrazole-3-carboxamide derivative” means a compound chosen from N-piperidino-5-(4-bromo-phenyl)-1-(2,4-dichlorophenyl)-4-ethylpyrazole-3-carboxamide and N-piperidino-5-(4-chlorophenyl)-1-(2,4-dichlorophenyl)-4-methylpyrazole-3-carboxamide. In the present description, these compounds are referred to as active principles according to the invention.

N-Piperidino-5-(4-bromophenyl)-1-(2,4-dichlorophenyl)-4-ethylpyrazole-3-carboxamide, referred to hereinbelow as compound A, is described in European patent EP-B-1 150 961. N-Piperidino-S-(4-chlorophenyl)-1-(2,4-dichlorophenyl)-4-methylpyrazole-3-carboxamide, referred to hereinbelow as compound B, the international non-proprietary name of which is rimonobant, is described in European patent EP-B-656 354. These compounds are cannabinoid CB₁ receptor antagonists.

These compounds are molecules that are very sparingly soluble in water, respectively: 0.1 μg/ml and 1 μg/l at pH 6.5. Furthermore, these compounds have high membrane permeability coefficients: respectively, 78×10⁻⁷ cm/s and 96×10⁻⁷ cm/s on the CaCO₂ cell model, as described by M. C. Gres et al in Pharmaceutical Research, 1198, 15(5), 726-7333.

A pharmaceutical composition containing a pyrazole-3-carboxamide derivative in micronized form and a surfactant wetting agent has been described in European patent EP-B-969 832. A pharmaceutical composition containing compound B mixed with Poloxamer 127 and a macrogolglyceride is described in international patent application WO 98/43635.

International Patent application WO 2004/009057 describes a process for preparing a dispersion of crystalline nanoparticles in an aqueous medium and the use of surfactant at a low concentration, making it possible to avoid the dissolution of the said nanoparticles; implementation examples especially concern compound A and compound B.

Pharmaceutical compositions have now been found, containing a pyrazole-3-carboxamide derivative according to the invention, which make it possible to improve the dissolution of the active principles according to the invention and the bioavailability to man in the fasted state.

These pharmaceutical compositions consist of a water-dispersible homogeneous mixture, in which the active principle according to the invention is dissolved in a lipid solvent to which is added a hydrophilic surfactant in order to spontaneously form a fine emulsion or a microemulsion during their dilution in aqueous medium; these compositions are known as self-emulsifying or self-microemulsifying compositions.

A microemulsion is a thermodynamically stable transparent system (Microemulsion and related system in Surfactant Sciences Series, Marcel Dekker Inc., 1988, 30, pp. 25-26).

The term “fine emulsion” means an emulsion in which the size of the dispersed globules is less than 5 μm. This fine emulsion is characterized in that it is stable enough to survive in the gastrointestinal tract up to the site of absorption, i.e. in the intestine.

All of the references described herein are incorporated herein by reference in their entirety.

### SUMMARY OF THE INVENTION

Thus, the present invention relates to a pharmaceutical composition in liquid or semi-solid form, which is self-emulsifying or self-microemulsifying in aqueous medium, for the oral administration of a pyrazole-3-carboxamide derivative chosen from: N-piperidino-5-(4-bromophenyl)-1-(2,4-dichlorophenyl)-4-ethylpyrazole-3-carboxamide and N-piperidino-5-(4-chlorophenyl)-1-(2,4-dichlorophenyl)-4-methylpyrazole-3-carboxamide, in which the said pyrazole-3-carboxamide derivative is dissolved in a mixture containing one or more lipid solvents for the pyrazole-3-carboxamide derivative and a nonionic hydrophilic surfactant whose hydrophilic/lipophilic balance is greater than 10 and preferably between 10 and 18.

### DETAILED DESCRIPTION OF THE INVENTION

According to the present invention, the weight proportion of the active principle is between 0.1 and 6%, preferably between 0.1 and 5%.

In the pharmaceutical composition according to the invention, the weight proportion of the lipid solvent or of the mixture of lipid solvents is from 35% to 75% and preferably 35% to 55%.

Preferably, the mixture of the pharmaceutical composition according to the invention also contains an amphiphilic cosolvent or a mixture of amphiphilic cosolvents. The presence of such an amphiphilic cosolvent promotes the dissolution of the active principle according to the invention and the subsequent emulsification of the pharmaceutical composition in aqueous medium. When it is present, the amphiphilic cosolvent, or each of the amphiphilic cosolvents, is in a weight proportion of less than 30%. When two amphiphilic solvents are present, they are in a total weight proportion of less than 50% and preferably less than 45%.

Thus, the pharmaceutical composition according to the present invention preferably contains from 10% to 50% and more particularly from 10% to 45% of amphiphilic cosolvent(s).

Preferably, the nonionic hydrophilic surfactant consists either of a single surfactant whose hydrophilic/lipophilic balance is greater than 10, or of a mixture of surfactants, the hydrophilic/lipophilic balance of the said mixture being greater than 10. According to the present invention, the surfactant is in a weight proportion of from 5% to 50%, preferably from 5% to 25% and optimally from 5% to 15%.

Thus, the concentration of surfactant used according to the present invention is markedly higher than the critical micelle concentration (CMC), so as to exploit the solubilizing capacity of the said surfactant under the conditions of the present invention.

The pharmaceutical compositions according to the present invention may be administered in soft gelatin capsules or in sealed or film-coated hard gelatin capsules.

According to the present invention, it is possible to use nonionic surfactants such as:

Polyoxyethylene 35 hydrogenated castor oil: Cremophor® EL, Polyoxyethylene 40 hydrogenated castor oil: Cremophor® RH40, both sold by BASF;

Polyoxyethylene polysorbate: Tween® 80, Tween® 20, Tween® 60, Tween® 85, sold by ICI;

Sorbitan monolaurate: Span 20, Sorbitan monooleate: Span 80, both sold by ICI;

Vitamin E/TPGS: Tocopheryl propylene glycol 1000 succinate, sold by Eastman;

Polyethylene glycol 15 hydroxystearate: Solutol® HS15, sold by BASF.

The preferred hydrophilic surfactants, alone or as a mixture, are Cremophor® RH40, Cremophoro EL, vitamin E TPGS and Tween 80.

Since surfactants such as the Span products are lipophilic, they are used as a mixture with other surfactants such that the hydrophilic/lipophilic balance of the surfactant mixture is greater than 10.

The terms “lipid solvents” and “amphiphilic cosolvents” mean natural fatty acid derivatives, preferably of plant origin, obtained by esterification with an alcohol:

either glycerol (mono-, di- or triglycerides),

or a glycol, optionally a long-chain glycol (macrogolglycerides).

Depending on the chain length of the fatty acid and the nature of the alcohol, these solvents have a more or less amphiphilic nature.

According to the present invention, it is possible to use lipid solvents such as:

Oleoyl macrogol 6 glycerides (polyglycosylated unsaturated glycerides): Labrafil® 1944 CS, sold by Gattefossé.

Propylene glycol caprylate caprate: Labrafac® PG, sold by Gattefossé.

Propylene glycol caprylic acid monoester: Capmul® PG-8, sold by Abitec.

Glyceryl oleate: Peceol® sold by Gattefossé.

Medium-chain mono- and diglycerides (capric caprylic): Capmul® MCM, sold by Abitec.

Polyglycerol oleate: Plurol® oleic, sold by Gattefossé.

Caprylic/capric triglyceride: Miglyol® 812, sold by Dynamit Nobel, Labrafac® CC, sold by Gattefossé.

The preferred lipid solvents, alone or as a mixture, are Labrafil® 1944 CS and Miglyol® 812 or Labrafac® CC or Capmul® MCM.

According to the present invention, amphiphilic cosolvents may be used, such as:

Propylene glycol monolaurate: Capmul® PGI2 sold by Abitec.

Propylene glycol monolaurate: Lauroglycol® 90, sold by Gattefossé.

Caprylocaproyl macrogol 8 glycerides: (ethyldiglycosylated saturated glycerides): Labrasol®, Gelucire 44-14 sold by Gattefossé, Diethylene glycol monoethyl ether: Transcutol®, sold by Gattefossé.

PEG 400: Polyethylene glycol 400, sold by Huls or ICI.

The preferred amphiphilic solvents, alone or as a mixture, are Labrasol and Gelucire 44-14, Capmul® PG12 or Lauroglycol® 90.

### EXAMPLES

Several pharmaceutical compositions according to the invention are prepared by using the following procedure: the chosen lipid solvent(s) and the surfactant are mixed together at a temperature of between 30 and 65° C. and preferably between 40 and 45° C., with stirring; this is performed after having melted the various solvents, if necessary. The active principle is incorporated while maintaining the stirring for the time required to dissolve the said active principle, and the formulation thus obtained is then transferred to the station for placing in gel capsules.

### Example 1

### Formulations with Lipid Solvent(s) and Surfactant

| Formulation | | |
|---|---|---|
| Example | Components | Weight % |
| 1.1 | Miglyol 812 | 49.6 |
| | Vitamin E TPGS | 49.6 |
| | Compound A | 0.8 |
| 1.2 | Miglyol 812 | 74.4 |
| | Vitamin E TPGS | 24.8 |
| | Compound A | 0.8 |

### Example 2

### Formulations with Lipid Solvent, Surfactant and Amphiphilic Cosolvent(s)

| Formulation | | |
|---|---|---|
| Example | Components | Weight % |
| 2.1 | Miglyol 812 | 45.9 |
| | Cremophor RH 40 | 12 |
| | Lauroglycol 90 | 21.5 |
| | Labrasol | 20 |
| | Compound A | 0.6 |
| 2.2 | Miglyol 812 | 41.5 |
| | Cremophor RH 40 | 12 |
| | Labrasol | 20 |
| | Lauroglycol 90 | 21.5 |
| | Compound B | 5 |
| 2.3 | Miglyol 812 | 45.25 |
| | Vitamin E TPGS | 12 |
| | Labrasol | 20 |
| | Lauroglycol 90 | 21.5 |
| | Compound A | 1.25 |
| 2.4 | Labrafil 1944 CS | 53.8 |
| | Tween 80 | 9.8 |
| | Labrasol | 17.9 |
| | Lauroglycol 90 | 17.9 |
| | Compound A | 0.6 |
| 2.5 | Labrafil 1944 CS | 70 |
| | Cremophor RH 40 | 9.5 |
| | Span 20 | 2.4 |
| | Labrasol | 17.5 |
| | Compound A | 0.6 |
| 2.6 | Labrafil 1944 CS | 39.8 |
| | Tween 85 | 49.7 |
| | Labrasol | 10 |
| | Compound B | 0.5 |
| 2.7 | Miglyol 812 | 41.5 |
| | Cremophor RH 40 | 12 |
| | Labrasol | 20 |
| | Lauroglycol 90 | 21.5 |
| | Compound A | 5 |
| 2.8 | Labrafac CC | 45.9 |
| | Cremophor EL | 12 |
| | Labrasol | 20 |
| | Lauroglycol 90 | 21.5 |
| | Compound A | 0.6 |
| 2.9 | Miglyol 812 | 41.5 |
| | Cremophor RH 40 | 12 |
| | Gelucire 44-14 | 20 |
| | Lauroglycol 90 | 21.5 |
| | Compound B | 5 |
| 2.10 | Miglyol 812 | 41.5 |
| | Cremophor RH 40 | 12 |
| | Labrasol | 20 |
| | Lauroglycol 90 | 11 |
| | Capmul MCM | 10.5 |
| | Compound B | 5 |
| 2.11 | Miglyol 812 | 41.5 |
| | Cremophor RH 40 | 12 |
| | Gelucire 44-14 | 20 |
| | Lauroglycol 90 | 21.5 |
| | Compound A | 5 |
| 2.12 | Miglyol 812 | 41.5 |
| | Cremophor RH 40 | 12 |
| | Labrasol | 20 |
| | Lauroglycol 90 | 15.5 |
| | Capmul MCM | 6 |
| | Compound B | 5 |

The ability to form a fine and stable emulsion is evaluated for each of the above formulations by diluting them tenfold in a simulated intestinal medium of pH 6.

Firstly, the time to the start of decantation, which indicates the stability of the emulsion, and secondly, under a microscope, the size of the oily globules dispersed in the aqueous phase, to control its fineness, are observed.

In all cases, the time to the start of decantation largely exceeds 24 hours.

The globules that are visible by optical microscopy often have a diameter of about 1 micron, the largest possibly being up to 5 microns.

Measurement of the in vitro dissolution kinetics:

The dissolution kinetics are studied in a paddle machine (machine No 2 of the Pharmacopoeia) in a simulated physiological medium of pH 6, at 37° C., and with stirring at 75 rpm.

The gel capsule formulation is introduced into the dissolution machine at time 0 and the percentage of finely emulsified product is determined at times 15, 30, 60 minutes and then 2, 3 and 4 hours by HPLC assay of the dissolution medium, after filtration through 5 μm. (This ensures that only the active principle that is in the form of a sufficiently fine emulsion, so as not to be retained by the 5 μm filter, is assayed).

The time of the experiment is longer than that required to reach the intestine (2 to 3 hours), which is the main site of absorption.

For comparative purposes, the same dissolution test was performed with a reference formulation, described below:

Reference formulation:

| Components | mg/unit |
|---|---|
| Compound A | 10 |
| Maize starch | 80 |
| Lactose monohydrate 200 mesh | 274 |
| Hypromellose | 10 |
| Sodium lauryl sulfate | 2 |
| Purified water | qs |
| Sodium croscarmellose | 20 |
| Magnesium stearate | 4 |
| Size No 0 gel capsule | gel capsule filled |
| | with 400 mg |

The experiment is performed with the same initial concentration of compound A in the medium for each dissolution test. Thus, in 250 ml of dissolution medium is placed one reference gel capsule containing 10 mg of compound A, i.e. 10 mg of compound A originating from the gel capsule prepared either from formulation 1.1 or from formulation 2.1.

TABLE 1
in vitro Dissolution

| | Time in minutes | | | | | |
|---|---|---|---|---|---|---|
| | 15 | 30 | 60 | 120 | 180 | 240 |
| % of Compound A in | 94.2 | 95.3 | 92.2 | 95 | 92.5 | 91.2 |
| fine emulsion with | | | | | | |
| formulation 1.1 | | | | | | |
| % of Compound A in | 33.6 | 82.5 | 97 | 99.7 | 100 | 99.6 |
| fine emulsion with | | | | | | |
| formulation 2.1 | | | | | | |
| % of Compound A | 14.2 | 23 | 23.8 | 26.7 | 20 | 22.7 |
| dissolved with the | | | | | | |
| reference formulation | | | | | | |

It is found that the formulations according to the invention make it possible to dissolve via the fine emulsion more than 80% of the compound according to the invention in 30 minutes, and that this dissolved state persists for at least 4 hours; in contrast, the reference formulation allows only about 25% of the active principle to be dissolved.

Measurements of Bioavailability in Man:

The pharmaceutical compositions according to the invention were also evaluated in vivo in man in order to study the influence of the formulation according to the invention on the bioavailability of the active principle in the fasted state and in the fed state.

In a first test, the bioavailability of the active principle in the fasted versus fed state was compared for the reference formulation described above.

In this test, a dose of 50 mg of compound A is administered orally, in a single intake, to 12 healthy volunteers, and the two administrations in the fasted and fed states are performed in a randomized manner and with an interval of 21 days.

Blood samples are taken after administration at times: 30 minutes, 1 hour, 1.5 hours, 2 hours, 2.5 hours, 3 hours, 4 hours, 6 hours, 8 hours, 12 hours, 24 hours, 36 hours, 48 hours, 72 hours, 120 hours and 168 hours. The various pharmacokinetic parameters allowing the bioavailability of the active principle to be established are measured.

TABLE 2
Bioavailability of compound A with the reference formulation

| | Tmax | Cmax | AUC |
|---|---|---|---|
| | (hours) | ng/ml) | (ng · h/ml) |
| Fed | 4 (2.5-6.0) | 524 (152) | 15949 (6192) |
| Fasted | 2.0 (1.0-4.0) | 126 (60) | 4480 (1542) |

With the reference formulation, it is found that the values of Cmax and AUC (area under the curve) are, respectively, 4.3 and 3.5 times greater for the fed individuals than for the fasted individuals.

In a second test, the bioavailability of the active principle was evaluated with the formulations according to the present invention.

In this test, a dose of 10 mg of compound A is administered to 12 fasted healthy volunteers, as a single oral intake, either with the reference formulation, with formulation 1.1 in a hard gelatin capsule, or with formulation 2.1 in a soft gelatin capsule. The administrations are performed in a randomized manner, with an interval of eight days. Blood samples are taken as in the preceding test, and the pharmacokinetic parameters are measured.

The results show an improvement in the bioavailability of the active principle in the fasted state, compared with the results obtained with the reference formulation.

After an interval of 15 days, formulation 2.1 was administered under the same conditions, to the same fed patients, and the pharmacokinetic parameters were measured.

TABLE 3
Absorption of compound A in healthy fasted individuals

| | Reference | Formulation | Formulation | Formulation |
|---|---|---|---|---|
| | formulation | 2.1 | 1.1 | 2.1 |
| | administered | administered | administered | administered |
| | while fasted | while fasted | while fasted | while fed |
| Cmax | 47 (17) | 160 (38) | 147 (33) | 139 (37.7) |
| ng/ml | | | | |
| Tmax | 1.5 (1.3) | 1 (1.2) | 1.5 (1.3) | 1.5 (0.5) |
| hours | | | | |
| AUC | 906 (420) | 1520 (664) | 1350 (497) | 1760 (782) |
| ng · h/ml | | | | |

The values in parentheses ( ) indicate the standard deviations.

With the formulations according to the invention, it is found that the Cmax and AUC values are similar irrespective of the formulation when the individual is fasted.

The improvement in the bioavailability in the fasted state, based on the increase in the AUC, is respectively 165% and 152% for formulations 2.1 and 1.1 with respect to the reference formulation.

Furthermore, with formulation 2.1, it is seen that the difference in bioavailability between the fasted state and the fed state is no longer significant.

Thus, the formulations according to the invention make it possible to significantly improve the bioavailability in the fasted state, thus leading to elimination of the difference in bioavailability between the fed state and the fasted state.

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

1. A pharmaceutical composition comprising either in liquid or semi-solid form, which is self-emulsifying or self-microemulsifying in aqueous medium, for the oral administration of a pyrazole-3-carboxamide derivative chosen from: N-piperidino-5-(4-bromophenyl)-1-(2,4-dichlorophenyl)-4-ethylpyrazole-3-carboxamide or N-piperidino-5-(4-chlorophenyl)-1-(2,4-dichlorophenyl)-4-methylpyrazole-3-carboxamide, in which said pyrazole-3-carboxamide derivative is dissolved in a mixture containing one or more lipid solvents for the pyrazole-3-carboxamide derivative in a weight proportion of from about 35% to about 75%, and a nonionic hydrophilic surfactant whose hydrophilic/lipophilic balance is between from about 10 and about 18, and said surfactant is present in a weight proportion of from about 5% to about 50%.

2. The pharmaceutical composition according to claim 1 further comprising an amphiphilic cosolvent or a mixture of amphiphilic cosolvents.

3. The pharmaceutical composition according to claim 2, wherein the amphiphilic cosolvent or each of the amphiphilic cosolvents present is in a weight proportion of less than about 30%.

4. The pharmaceutical composition according to claim 2, wherein the amphiphilic cosolvent or the mixture of amphiphilic cosolvents is in a weight proportion of between about 10% and about 50%.

5.-8. (canceled)

9. The pharmaceutical composition according to claim 1, wherein the lipid solvent or the mixture of lipid solvents is in a weight proportion of from about 35% to about 55%.

10. The pharmaceutical composition according to claim 2, wherein the lipid solvent or the mixture of lipid solvents is in a weight proportion of from about 35% to about 55%.

11. The pharmaceutical composition according to claim 3, wherein the lipid solvent or the mixture of lipid solvents is in a weight proportion of from about 35% to about 55%.

12. The pharmaceutical composition according to claim 4, wherein the lipid solvent or the mixture of lipid solvents is in a weight proportion of from about 35% to about 55%.

13. The pharmaceutical composition according to claim 1, wherein the surfactant consists of a single surfactant or of a mixture of surfactants, the hydrophilic/lipophilic balance of which is between about 10 and about 18.

14. The pharmaceutical composition according to claim 2, wherein the surfactant consists of a single surfactant or of a mixture of surfactants, the hydrophilic/lipophilic balance of which is between about 10 and about 18.

15. The pharmaceutical composition according to claim 3, wherein the surfactant consists of a single surfactant or of a mixture of surfactants, the hydrophilic/lipophilic balance of which is between about 10 and about 18.

16. The pharmaceutical composition according to claim 4, wherein the surfactant consists of a single surfactant or of a mixture of surfactants, the hydrophilic/lipophilic balance of which is between about 10 and about 18.

17. The pharmaceutical composition according to claim 9, wherein the surfactant consists of a single surfactant or of a mixture of surfactants, the hydrophilic/lipophilic balance of which is between about 10 and about 18.

18. The pharmaceutical composition according to claim 10, wherein the surfactant consists of a single surfactant or of a mixture of surfactants, the hydrophilic/lipophilic balance of which is between about 10 and about 18.

19. The pharmaceutical composition according to claim 1, wherein the surfactant consists of a single surfactant or of a mixture of surfactants, the hydrophilic/lipophilic balance of which is between about 10 and about 18.

20. The pharmaceutical composition according to claim 12, wherein the surfactant consists of a single surfactant or of a mixture of surfactants, the hydrophilic/lipophilic balance of which is between about 10 and about 18.

21.-24. (canceled)

25. The pharmaceutical composition according to claim 1, wherein the surfactant is in a weight proportion of from about 5% to about 15%.

26. The pharmaceutical composition according to claim 2, wherein the surfactant is in a weight proportion of from about 5% to about 15%.

27. The pharmaceutical composition according to claim 3, wherein the surfactant is in a weight proportion of from about 5% to about 15%.

28. The pharmaceutical composition according to claim 4, wherein the surfactant is in a weight proportion of from about 5% to about 15%.

29.-32. (canceled)

33. The pharmaceutical composition according to claim 1, wherein the pyrazole-3-carboxamide derivative is in a weight proportion of from about 0.1% to about 6%.

34. The pharmaceutical composition according to claim 2, wherein the pyrazole-3-carboxamide derivative is in a weight proportion of from about 0.1% to about 6%.

35. The pharmaceutical composition according to claim 3, wherein the pyrazole-3-carboxamide derivative is in a weight proportion of from about 0.1% to about 6%.

36. The pharmaceutical composition according to claim 4, wherein the pyrazole-3-carboxamide derivative is in a weight proportion of from about 0.1% to about 6%.

37. The pharmaceutical composition according to claim 1, which is administered in soft gelatin capsules.

38. The pharmaceutical composition according to claim 2, which is administered in soft gelatin capsules.

39. The pharmaceutical composition according to claim 3, which is administered in soft gelatin capsules.

40. The pharmaceutical composition according to claim 4, which is administered in soft gelatin capsules.

41. The pharmaceutical composition according to claim 1, which is administered in sealed or film-coated hard gelatin capsules.

42. The pharmaceutical composition according to claim 2, which is administered in sealed or film-coated hard gelatin capsules.

43. The pharmaceutical composition according to claim 3, which is administered in sealed or film-coated hard gelatin capsules.

44. The pharmaceutical composition according to claim 4, which is administered in sealed or film-coated hard gelatin capsules.

45. The pharmaceutical composition according to claim 1, wherein the lipid solvent is chosen from:
oleoyl macrogol 6 glycerides (unsaturated polyglycosyl glycerides);
propylene glycol caprylate caprate;
propylene glycol caprylic acid monoester;
glyceryl oleate;
medium-chain (capric capiylic) mono- and diglycelide;
polyglycerol oleate; and
caprylic/capric triglyceride; or
a mixture in any combination thereof.

46. The pharmaceutical composition according to claim 2, wherein the lipid solvent is chosen from:
oleoyl macrogol 6 glycerides (unsaturated polyglycosyl glycerides);
propylene glycol caprylate caprate;
propylene glycol caprylic acid monoester;
glyceryl oleate;
medium-chain (capric caprylic) mono- and diglycelide;
polyglycerol oleate; and
caprylic/capric triglyceride; or
a mixture in any combination thereof.

47. The pharmaceutical composition according to claim 3, wherein the lipid solvent is chosen from:
oleoyl macrogol 6 glycerides (unsaturated polyglycosyl glycerides);
propylene glycol caprylate caprate;
propylene glycol caprylic acid monoester;
glyceryl oleate;
medium-chain (capric caprylic) mono- and diglyceride;
polyglycerol oleate; and
caprylic/capric triglyceride; or
a mixture in any combination thereof.

48. The pharmaceutical composition according to claim 4, wherein the lipid solvent is chosen from:
oleoyl macrogol 6 glycerides (unsaturated polyglycosyl glycerides);
propylene glycol caprylate caprate;
propylene glycol caplylic acid monoester;
glyceryl oleate;
medium-chain (capric caprylic) mono- and diglyceride;
polyglycerol oleate; and
caprylic/capric triglyceride; or
a mixture in any combination thereof.

49. The pharmaceutical composition according to claim 1, wherein the surfactant is chosen from:
polyoxyethylene 35 hydrogenated castor oil;
polyoxyethylene 40 hydrogenated castor oil;
polyoxyethylene polysorbate;
sorbitan monolaurate;
vitamin E/TPGS: tocopherol propylene glycol 1000 succinate; and
polyethylene glycol 15 hydroxystearate; or
a mixture in any combination thereof.

50. The pharmaceutical composition according to claim 2, wherein the surfactant is chosen from:
polyoxyethylene 35 hydrogenated castor oil;
polyoxyethylene 40 hydrogenated castor oil;
polyoxyethylene polysorbate;
sorbitan monolaurate;
vitamin E/TPGS: tocopherol propylene glycol 1000 succinate; and
polyethylene glycol 15 hydroxystearate; or
a mixture in any combination thereof.

51. The pharmaceutical composition according to claim 3, wherein the surfactant is chosen from:
polyoxyethylene 35 hydrogenated castor oil;
polyoxyethylene 40 hydrogenated castor oil;
polyoxyethylene polysorbate;
sorbitan monolaurate;
vitamin E/TPGS: tocopherol propylene glycol 1000 succinate; and
polyethylene glycol 15 hydroxystearate; or
a mixture in any combination thereof.

52. The pharmaceutical composition according to claim 4, wherein the surfactant is chosen from:
polyoxyethylene 35 hydrogenated castor oil;
polyoxyethylene 40 hydrogenated castor oil;
polyoxyethylene polysorbate;
sorbitan monolaurate;
vitamin E/TPGS: tocopherol propylene glycol 1000 succinate; and
polyethylene glycol 15 hydroxystearate; or
a mixture in any combination thereof.

53. The pharmaceutical composition according to claim 2, wherein the amphiphilic cosolvent is chosen from:
caprylocaproyl macrogol 8 glycerides or LABRASOL®;
lauroyl macrogoglyceride or GELUCIRE® 44-14; and
propylene glycol monolaurate or CAPMUL® PG12 or LAUROGLYCOL® 90; or
a mixture in any combination thereof.

54. A pharmaceutical composition comprising:

| | |
|---|---|
| caprylic/capric triglyceride | 45.9%; |
| polyoxyethylene 40 hydrogenated castor oil | 12%; |
| propylene glycol monolaurate | 21.5%; |
| caprylocaproyl macrogol 8 glycerides | 20%; and |
| N-piperidino-5-(4-bromophenyl)-1-(2,4- | 0.6%. |
| dichlorophenyl)-4-ethylpyrazole-3-carboxamide | |

55. A pharmaceutical composition comprising:

| | |
|---|---|
| caprylic/capric triglyceride | 41.5%; |
| polyoxyethylene 40 hydrogenated castor oil | 12%; |
| propylene glycol monolaurate | 21.5%; |
| caprylocaproyl macrogol 8 glycerides | 20%; and |
| N-piperidino-5-(4-bromophenyl)-1-(2,4- | 5%. |
| dichlorophenyl)-4-ethylpyrazole-3-carboxamide | |

56. A pharmaceutical composition comprising:

| | |
|---|---|
| caprylic/capric triglyceride | 41.5%; |
| polyoxyethylene 40 hydrogenated castor oil | 12%; |
| propylene glycol monolaurate | 21.5%; |
| caprylocaproyl macrogol 8 glycerides | 20%; and |
| N-piperidino-5-(4-chlorophenyl)-1-(2,4- | 5%. |
| dichlorophenyl)-4-methylpyrazole-3-carboxamide | |
